(19) United States
(12) Patent Application Publication
(10) Pub. No.: US 2023/0355847 A1
(43) Pub. Date: Nov. 9, 2023

(54) VASCULAR PORT SYSTEM INCLUDING AN INTERCHANGEABLE STENT CARTRIDGE, AND METHOD THEREOF

(71) Applicant: Zeno Yeates, New Orleans, LA (US)

(72) Inventor: Zeno Yeates, New Orleans, LA (US)

(21) Appl. No.: 17/739,720

(22) Filed: May 9, 2022

(51) Int. Cl. A61L 31/16; A61F 2/90; A61L 31/10; A61F 2/06

(52) U.S. Cl. CPC A61L 31/16; A61F 2/90; A61L 31/10; A61F 2/06; A61F 2230/0006; A61F 2250/0067

(57) ABSTRACT

A medical vascular port and method thereof for directing light, electric voltages, or magnetic fields onto an interchangeable stent with a monolayer of genetically modified endothelial cells on the interior surface. Genetic mechanisms within the cells are triggered by light, voltage, or magnetic fields to produce therapeutic proteins or peptides thereby secreted into the bloodstream. The vascular port system includes a vascular stent having a main body with a nozzle at each end. The sealable vascular port has a cap and contains the vascular stent inside. The vascular port has a pair of tubes connected to the nozzles of the vascular stent and connects to cavities in the vascular port. A second pair of tubes connects to the cavities and descends through the bottom of the vascular port to engage with an artery/vein. The vascular port system includes a controllable light source, a voltage source, or a magnetic field source.

### CROSS-REFERENCE TO RELATED APPLICATIONS

Not applicable.

### RELATED CO-PENDING U.S. PATENT APPLICATIONS

Not applicable.

### INCORPORATION BY REFERENCE OF SEQUENCE LISTING PROVIDED AS A TEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to vascular stents. More particularly, certain embodiments of the invention relate to an implantable vascular port comprising an interchangeable stent-cartridge system.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. Optogenetics refers to a technique by which genes producing light sensitive proteins are engineered into a cell line so as to create a functional environment in which light can be used to stimulate a change within the cell. Although the field of optogenetics has a primary focus on neurobiology, its components could theoretically be applied to any tissue type or anatomical structure. Embodiments of the present invention may be employed in applications concerning the use of optogenetic mechanisms that activate transcription of genes. A set of optogenetic systems already exists composed of protein fusions between a light sensitive protein, a transactivator protein, and a transcription factor. Transactivator protein refers to any protein activator such as VP16, MyoD, or FoxA (Hirai, Hiroyuki, et al. “Structure and Functions of Powerful Transactivators: VP16, MyoD and FoxA.” The International Journal of Developmental Biology, doi:10.1387/ijdb.103194hh.) for example that localizes to a gene promoter region and then recruits the transcriptional machinery to drive strong expression of the downstream gene. Transcription factor refers to any protein that binds to specific promoter regions of DNA and initiates and regulates transcription of the gene downstream from the promoter. Light sensitive protein refers to any protein that undergoes conformational change when exposed to light of a certain wavelength and thus assumes a particular catalytic activity. An example of an optogenetic system would be the system devised by Dr. Kevin Gardner (Gardner, K., Motta-Mena, L., Zoltowski, B. (2012) U.S. Patent Number 20170204421A1. Washington, DC: U.S. Patent and Trademark Office.) in which the activator protein VP16 is fused to a DNA binding domain and a Light Oxygen Voltage (LOV) domain as well as a nuclear localization sequence. (Motta-Mena, Laura B, et al. “An Optogenetic Gene Expression System with Rapid Activation and Deactivation Kinetics.” Nature Chemical Biology, March 2014, pp. 196-202., doi:10.1038/nchembio.1430.)

In addition, voltage-gated membrane channels such as the calcium channel can be used to drive gene expression by relying on transcription factors endogenous to the cell such as NFAT which bind calcium, undergo conformation change, and then bind to a synthetic NFAT promoter which can be placed in front of the gene of interest. (Xie, Mingqi, et al. β-Cell—Mimetic Designer Cells Provide Closed-Loop Glycemic Control.” Science, vol. 354, 9 Dec. 2016, pp. 1296-1301., doi:10.1126/science. aaf4006.) Such channels are triggered by application of an electrical impulse to the cell membrane over a specific voltage threshold. For example, calcium channels are known to have an activation threshold of −30 mV. (Niisato, Naomi. “Ion Transport: Calcium Channels.” Biomedical Sciences, edited by Yoshinori Marunaka, Elsevier, 2020.)

In addition, it has been shown that electromagnetic fields with magnitude of 8 μT and frequency of 60 Hz can stimulate gene expression through certain Electromagnetic Field Response Elements (EMRE's) which are found in the promoter region of genes C-MYC as well as HSP70. (Lin, H., et al. “Regulating Genes with Electromagnetic Response Elements.” Journal of Cellular Biochemistry, 2001, pp. 143-148.) The response was shown to be dependent upon the inclusion of multiple nCTCTn nucleic acid repeats, where n is any nucleic acid, C is cytosine, and T is thymine.

Tubular therapeutic cellular graft stents exist for the purpose of eluting drugs into the bloodstream (Spielberg, Theodore. (2002) U.S. Patent Number U.S. Pat. No. 7,044,965B1 Washington, DC: U.S. Patent and Trademark Office.) and the genetic engineering of endothelial cells for the purpose of adhering to a vascular stent has been explored. (Flugelman, M. Y., et al. “Genetically Engineered Endothelial Cells Remain Adherent and Viable after Stent Deployment and Exposure to Flow in Vitro.” Circulation Research, February 1992, pp. 348-354., doi: 10.1161/01.res.70.2.348., Dichek, D. A., et al. “Seeding of Intravascular Stents with Genetically Engineered Endothelial Cells.” Circulation, vol. 80, no. 5, 1 Nov. 1989, pp. 1347-1353.) Vascular endothelial cells are known to naturally secrete large proteins such as Von Willebrand Factor (Wei, Haoche, et al. “Characterization of the Polarized Endothelial Secretome.” The FASEB Journal, vol. 33, no. 11, 2019, pp. 12277-12287., doi:10.1096/fj.201900262r), so the secretory pathway is fully operational in such cells.

A variety of infusion ports currently exist for the purpose of dialysis and administering medication, and traditionally a central venous catheter may be implanted into the chest to feed into the subclavian vein. (Marino, Paul. Marino's ICU Book. 4th ed., LWW, 2013.)

Simple inline microfluidic air bubble traps have also been developed as a means to remove air bubbles from solution passing through sections of tubing using a PTFE filter plate with holes drilled into either end with the tubing fitting into the holes and glued in place using Pattex Plastix glue and the tubing connection being strengthened with Araldite. (Van Lintel, Harald, et al. “High-Throughput Micro-Debubblers for Bubble Removal with Sub-Microliter Dead Volume.” Micromachines, 2012, pp. 218-224., doi:10.3390/mi3020218.) Often there is a flow restrictor connected in tandem with the air bubble trap in order to slow the flow of liquid through the trap thereby allowing more time for air bubbles to dissipate through the membrane.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIGS. 1A-1E illustrate an exemplary tubular stent, in accordance with an embodiment of the present invention. FIG. 1A is a side view of the tubular stent. FIG. 1B is a cross-sectional view of the tubular stent. FIG. 1C is a side view of an exemplary solenoid sheathing component. FIG. 1D is a side view of the tubular stent with the solenoid sheathing component in place, and FIG. 1E is a cross-sectional view of a variation of the tubular stent comprising fenestrations that form a three-dimensional lattice structure inside the stent;

FIG. 2 is a top view of the top of a vascular port, in accordance with an embodiment of the present invention;

FIGS. 3A and 3B illustrate the vascular port, in accordance with an embodiment of the present invention. FIG. 3A is a top internal view of the vascular port with a stent in place, and FIG. 3B is a top internal perspective view of the vascular port with a stent encompassed by a solenoid and metal end pieces in place;

FIG. 4 is a top internal view of the vascular port without a stent in place, in accordance with an embodiment of the present invention;

FIG. 5 is a side perspective view of internal circular disk and its components, in accordance with an embodiment of the present invention;

FIGS. 6A and 6B illustrate the vascular port, in accordance with an embodiment of the present invention. FIG. 6A is a side perspective view of the vascular port with the top screw cap in place, and FIG. 6B is a cross sectional view of the vascular port without the top screw cap;

FIG. 7 is a bottom view of the vascular port, in accordance with an embodiment of the present invention;

FIG. 8 is an interior bottom view of the top screw cap, in accordance with an embodiment of the present invention;

FIGS. 9A and 9B illustrate an exemplary vascular port, in accordance with an embodiment of the present invention. FIG. 9A is a cross sectional view of the vascular port with interior components installed, and FIG. 9B is an exploded view of the components situated within the vascular port;

FIG. 10 is an electronic schematic for a microcontroller connected to a metal, voltage/light operated version of an interchangeable stent for use in a vascular port, in accordance with an embodiment of the present invention;

FIG. 11 is an electronic schematic for a microcontroller connected to a solenoid/light operated interchangeable stent for use in a vascular port, in accordance with an embodiment of the present invention;

FIG. 12 is a perspective view of the vascular port implanted under skin and grafted to the vascular system, in accordance with an embodiment of the present invention;

FIGS. 13A and 13B illustrate an exemplary screw connector, in accordance with an embodiment of the present invention. FIG. 13A is an exterior perspective right side view of the screw connector, and FIG. 13B is an exterior perspective left side view of the screw connector;

FIG. 14 is a perspective view of a terminal end of the upper region of tubing which may be connected to a stent, in accordance with an embodiment of the present invention; and

FIGS. 15A and 15B illustrate an exemplary connection between tubing and a tubular stent, in accordance with an embodiment of the present invention. FIG. 15A is a side view of the junction of the terminal end of the tubing to the end nozzle of the main body of the tubular stent, and FIG. 15B is a partially transparent side view of the same junction.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said, “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred, or induced by any pattern(s) of description, embodiments, examples, or referenced prior-art that may (or may not) be provided in the present patent.

References to “end user”, or any similar term, as used herein, is generally intended to mean late-stage user(s) as opposed to early-stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):
“Comprising” And “contain” and variations of them—Such terms are open-ended and mean “including but not limited to”. When employed in the appended claims, this term does not foreclose additional structure or steps. Consider a claim that recites: “A memory controller comprising a system cache . . . .” Such a claim does not foreclose the memory controller from including additional components (e.g., a memory channel unit, a switch).
“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn.112, sixth paragraph, for that mechanism/unit/circuit/component. “Configured to” may also include adapting a manufacturing process to fabricate devices or components that are adapted to implement or perform one or more tasks.
“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus, in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s). Furthermore, any statement(s), identification(s), or reference(s) to a structure(s) and/or element(s) that corresponds to and/or supports a claim limitation(s) phrased in functional limitation terms covered by 35 USC § 112(6) (post AIA 112(f)) should be understood to be identified by way of example and not limitation, and as such, should not be interpreted to mean that such recited structure and/or element is/are the only structure(s) and/or element(s) disclosed in this patent application that corresponds to and/or supports such claim limitations phrased in functional limitation terms. This claims interpretation intention also applies to any such subsequent statements made by Applicant during prosecution.

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

Embodiments of the present invention may comprise a medical vascular port for directing light, electric voltages, or magnetic fields onto an interchangeable tubular stent cartridge with a monolayer of genetically modified endothelial cells on the interior surface of the stent such that genetic mechanisms within the cells can be triggered by the light, voltage, or said magnetic fields to produce therapeutic proteins or peptides that can then be secreted into the bloodstream. This port may be a self-contained and implantable vascular port system that contains the stent along with the electronics for modulating gene expression in the monolayer of cells within the stent. It is contemplated that since the stents may be made interchangeable, stents may be removed or replaced without requiring surgery each time a stent is removed or inserted. In some such embodiments, the port may be placed in the chest, abdomen, or peritoneum. It is contemplated that some embodiments may also seek to connect arterial blood flow to venous blood flow in the manner that arteriovenous (AV) grafts are typically performed. Although, connections from vein to vein or artery to artery may also be possible. AV grafting tubes are typically made of polytetrafluorourethylene (PTFE). (Berardinelli, L. “Grafts and Graft Materials as Vascular Substitutes for Haemodialysis Access Construction.” European Journal of Vascular and Endovascular Surgery, vol. 32, no. 2, August 2006, pp. 203-211.)

One embodiment may comprise an interchangeable vascular stent cartridge system, a light, voltage, or magnetic field source, and a vascular port that connects the stent to the vascular system. This embodiment may provide a method for applying light of various wavelengths, electricity of different magnitudes, or a magnetic field of different field strengths to a monolayer of endothelial cells or any other cell type that are contained within a tubular stent including stem cells, hybridoma cells, vascular smooth muscle cells, and fibroblasts. In various embodiments the stent may be made of plastic for light sensitive protein mechanisms (optogenetic purposes), metal for voltage sensitive protein mechanisms (voltage activated genetic responses), or plastic with solenoid coils around it for magnetically sensitive promoter regions (magnetic field activated genetic responses). The light, electric voltage, or magnetic field applied to the stent typically triggers a response from optogenetic protein mechanisms, voltage-gated membrane channels, or magnetically inducible promoters, which can then be used to alter transcription of different genes within the cell by placing a promoter adjacent to that gene which responds to light, responds to changes in ion concentration within the cell, or responds to changes in magnetic field strength. Thus, these embodiments may provide a method of activating genes inside the monolayer of cells within the stent by three different methods that in turn produce proteins that are secreted by the cells into the bloodstream.

Endothelial cells have a half-life of about 6 years on average and do not continuously divide as adult cells in the endothelium, but typically remain in a “quiescent” state. (Ricard, Nicolas, et al. “The Quiescent Endothelium: Signaling Pathways Regulating Organ-Specific Endothelial Normalcy.” Nature Reviews Cardiology, vol, 18, no. 8, 2021, pp, 565-580., https://doi.org/10.1038/s41569-021-00517-4). The use of an interchangeable cartridge system in embodiments of the present invention may be able to harness the lifespan of an endothelial cell monolayer, which can last multiple years, for therapeutic purposes. (D. Pearson, Jeremy. “Plasticity of Adult Endothelium: How Frequent and to What Extent?” Cardiovascular Research, vol. 108, December 2015, pp. 319-320.) Then, users may be able to readily and accessibly exchange stents at the end of the lifespan of the cells without repetitive surgical procedures. In addition, some embodiments may also provide a static and controlled environment for the timed release of proteins into the bloodstream and also a means to control duration and quantity of the secretion of the proteins from the cell monolayer within the stent based on the duration and intensity of the light, voltage, or magnetic field source therein. The fact that the adult endothelium does not continuously proliferate aids in creating a static environment for the delivery of peptides. That being said, the adult endothelial cells typically will not multiply out of control and obstruct the interior of the stent. Moreover, some embodiments can deliver therapeutic peptides directly to the bloodstream while also maintaining a division between genetically engineered cells and human cells. Endothelial cells are well known for naturally secreting a wide array of different peptides into the bloodstream, so they seem to be good candidates to be genetically engineered to secrete non-native peptides into the bloodstream as well. One can expect that virtually any peptide therapeutics (i.e., peptides that mimic antibodies, protein hormones, etc.) could be theoretically secreted from the stent. For example, without limitation, the process of phage display to select for peptides that bind to certain receptors would be a useful process that could be used in conjunction with a vascular stent port according to an embodiment of the present invention. Finally, it is contemplated that embodiments of the present invention may provide an enabling platform technology for a host of other future peptide therapeutics or even therapeutic viruses to be delivered to the body. For example, in one such embodiment, the stent could be used to secrete therapeutic viruses into the bloodstream, thereby creating a viable gene therapy delivery system. Other embodiments may be used for applications such as, but not limited to, General Constitutive Secretion of Peptide Therapeutics, in vivo infusion of drugs with fast a half life, etc.

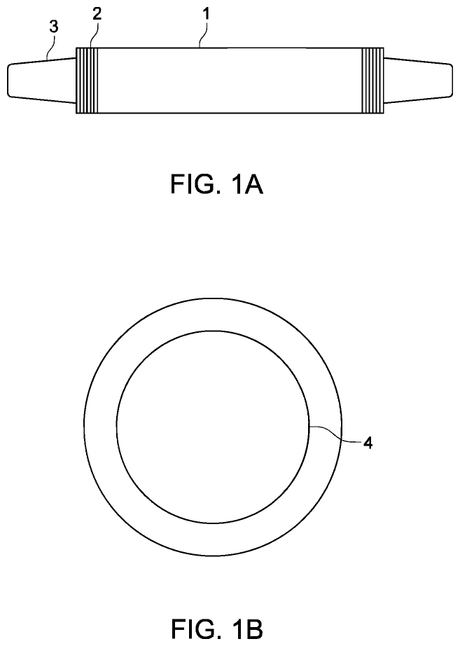

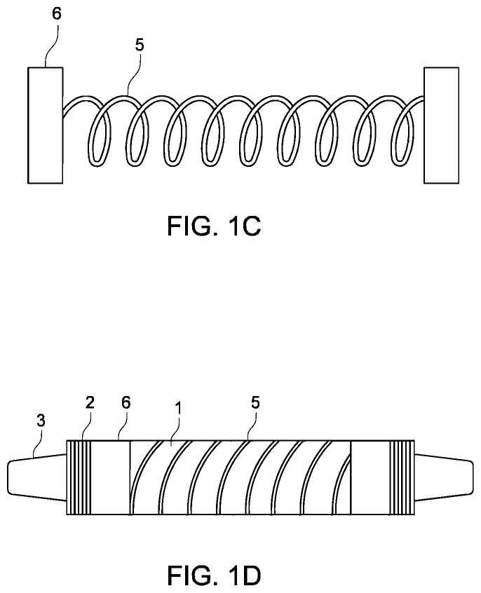

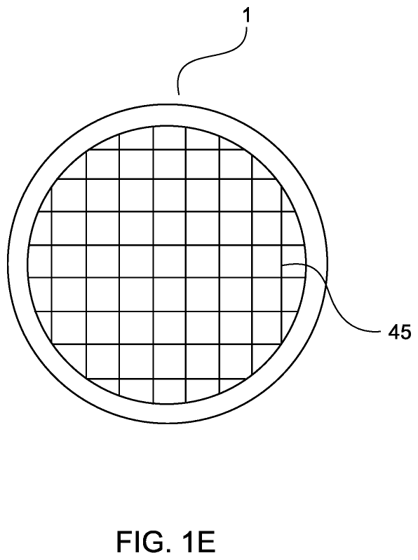

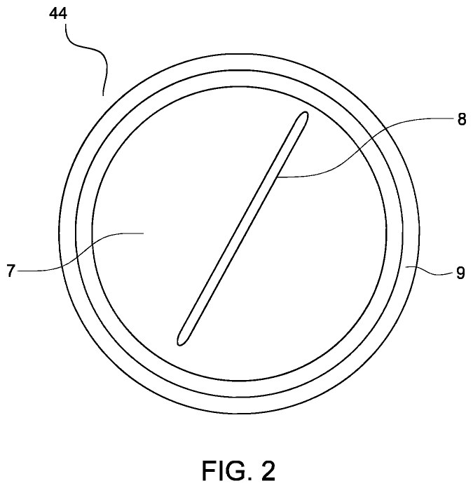

FIGS. 1A-1D illustrate an exemplary tubular stent, in accordance with an embodiment of the present invention. FIG. 1A is a side view of tubular stent. FIG. 1B is a cross-sectional view of tubular stent. FIG. 1C is a side view of an exemplary solenoid sheathing component. FIG. 1D is a side view of tubular stent with the solenoid sheathing component in place, and FIG. 1E is a cross-sectional view of a variation of the tubular stent comprising fenestrations 45 that form a three-dimensional lattice structure inside the stent. In the present embodiment, referring to FIG. 1A, FIG. 1B and FIG. 1D, tubular stent comprises a main body 1 with threaded regions 2 and end nozzles 3. It is contemplated that main body 1 may be made of plastic or metal and comprises a pair of threaded regions 2 on either end or an end nozzle 3 at each terminal end of the stent. The stent might also be made of a type of ceramic material that transmits electrical voltage and/or is transparent enough to transmit light. Some embodiments may employ use of a transparent ceramic, which when combined with a solenoid, will allow the contents in the stent to be subject manipulation by light, electrical voltage, and magnetic fluctuations. As shown in FIG. 1B, a monolayer of cells 4 that have genetic mechanisms that respond to changes in wavelength or intensity of light or changes in membrane potential such as, but not limited to, endothelial cells may be present on the interior surface of the stent. The interior surface of the stent may be made of high impact polystyrene modified by corona discharge in atmospheric conditions or gas plasma under vacuum conditions so as to make its functional groups more hydrophilic to help enable certain extracellular matrix proteins and thus cells to sufficiently attach to the interior surface of the stent. This method for treating polystyrene is widely used in cell culture dishes in the biotech industry for growing mammalian cells since it allows cells to secrete extracellular matrix attachment proteins that bind the cell to the plastic surface of the dish. In addition to having a modified polystyrene surface, it is traditional practice with respect to endothelial cells to “coat” the plastic surface with a solution of fibronectin at a 1:100 dilution ratio in Phosphate Buffered Saline and add this mixture to the plastic surface at a volume of 150 microliters per centimeter squared. The plastic surface is then allowed to incubate overnight in a carbon dioxide incubator at 37° C. Referring to FIG. 1C, the solenoid sheathing component comprises a solenoid 5 and a pair of cylindrical end pieces 6, which may be made of metal. The purpose of the end pieces, which are cylindrical, is to make contact with the voltage clamps of the vascular port thereby allowing electricity to flow through the solenoid. Referring to FIG. 1D, solenoid 5 and metal end pieces 6 are shown encompassing tubular stent body 1. In one embodiment of the present invention the stent body being encompassed by the solenoid may be made of plastic. However, other embodiments may be made of other appropriate materials, for example metal. Referring to FIG. 1E, in one embodiment the tubular stent may comprise uniform fenestrations 45 forming a three-dimensional lattice within the stent body 1 so as to increase the internal surface area in comparison to a completely hollow stent. This increased surface area provides additional area on which cells may grow within the stent and thus may increase the total amount of peptides possibly secreted from the stent.

FIG. 2 is a top view of the top of a vascular port 44, in accordance with an embodiment of the present invention. In the present embodiment, vascular port 44 comprises a top screw cap 7 that may be dome shaped and may be utilized to seal the top of vascular port 44 and its internal components from the outside environment. Top screw cap 7 comprises a groove 8 that serves as a means to aid in screwing open top screw cap 7 by twisting. On the outside of vascular port 44, a circular lip 9 helps to hold the port underneath the skin of a user in which vascular port 44 is implanted.

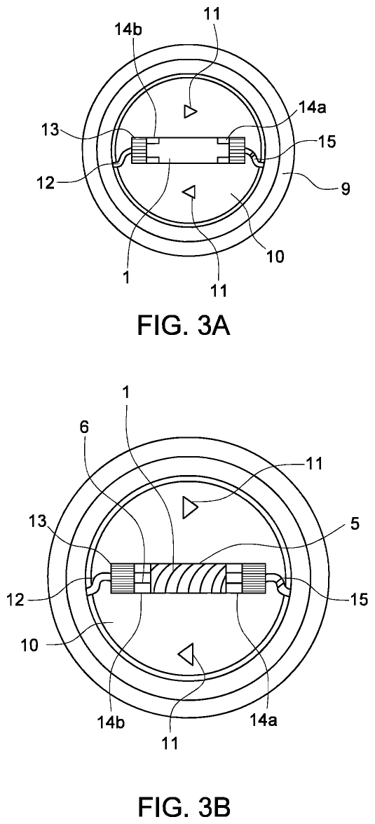

FIGS. 3A and 3B illustrate vascular port 44, in accordance with an embodiment of the present invention. FIG. 3A is a top internal view of vascular port 44 with a stent in place, and FIG. 3B is a top internal perspective view of vascular port 44 with a stent encompassed by solenoid 5 and end pieces 6 in place. As shown in FIG. 3A, tubular stent body 1 sits inside a pair of voltage clamps 14a and 14b that allow the stent to snap into place. Voltage clamps 14a and 14b are attached to an internal circular disk 10 which separates an upper portion of vascular port 44 from a bottom portion of vascular port 44. Tubing 12 is sized properly to connect to stent nozzles 3 and may be secured in place on either end of the main stent body 1 by a set of screw connectors 13 which screw into threaded regions 2 on the tubular stent body 1. Within internal circular disk 10, there are two triangular depressions 11 which serve as a means to assist in turning internal circular disk 10 in order to open vascular port 44 and access the bottom portion. A microfluidic filter bubble trap 15 as described in Van Lintel et al. may be placed within the upper region of tubing 12 connected to one end of the stent. Microfluidic filter bubble trap 15 may serve to generally prevent bubbles from entering the bloodstream when the upper region of tubing 12 is clamped at the end containing microfluidic air bubble trap 15 during the stent change procedure.

Referring to FIG. 3B, tubular stent body 1 encompassed by solenoid 5 and end pieces 6 may be held in place by voltage clamps 14a and 14b. It should be appreciated that, while shown as cylindrical, the phrase “tube” may indicate an elongated hollow conduit that may be the shape of a rectangular, hexagonal, or any other shape that serves as a conduit between two ends. Solenoid end pieces 6 may be situated in direct contact with the voltage clamps 14a and 14b which also serve the purpose of delivering a voltage to solenoid 5 through end pieces 6. In an embodiment where there is no solenoid sheathing component present, for example, without limitation, as illustrated in FIG. 3A, the stent snaps into voltage clamps 14a and 14b as a simple plastic or metal tube.

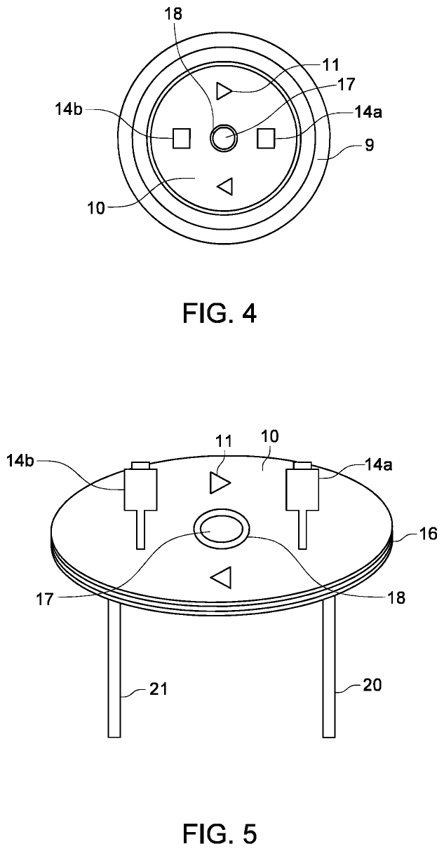

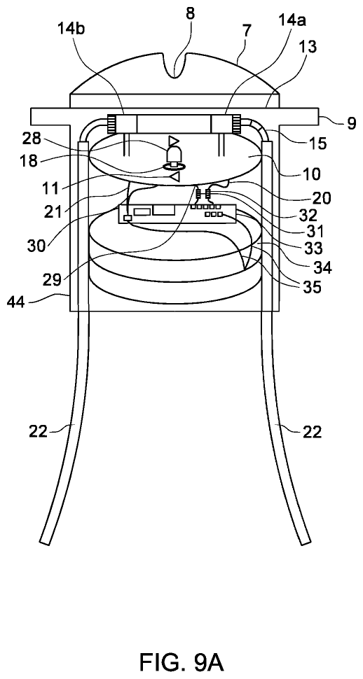

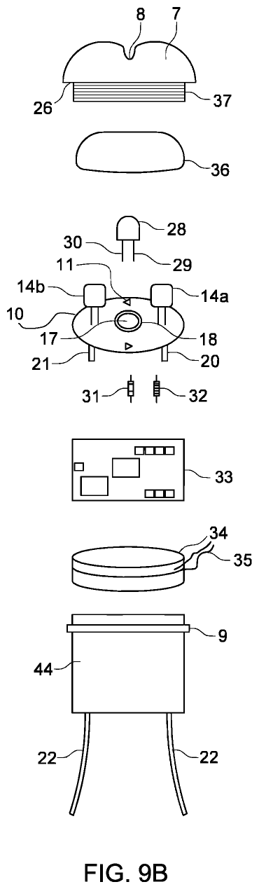

FIG. 4 is a top internal view of vascular port 44 without a stent in place, in accordance with an embodiment of the present invention. FIG. 5 is a side perspective view of internal circular disk 10 and its components, in accordance with an embodiment of the present invention. As shown in FIG. 4 and FIG. 5, there is a hole 17 in the center of internal circular disk 10 as well as a circular depression 18 that surrounds hole 17 in the center of circular disk 10. Referring to FIG. 5, a set of wires 20, 21 may descend from voltage clamps 14a and 14b respectively which connect to electronics within vascular port 44 as shown by way of example in FIGS. 9A and 9B. Threading 16 is disposed around the circumference of internal circular disk 10, which may enable circular disk 10 to be threaded into place within vascular port 44.

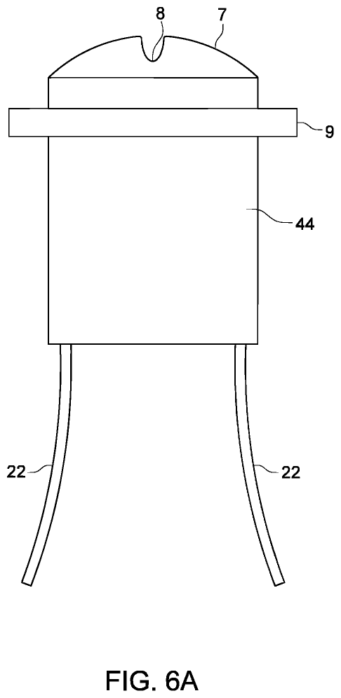

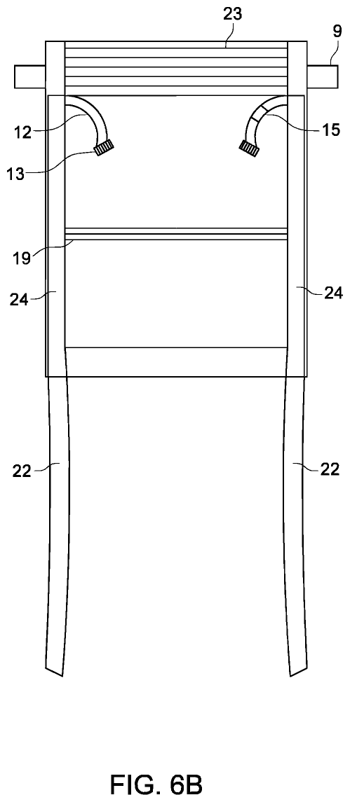

FIGS. 6A and 6B illustrate vascular port 44, in accordance with an embodiment of the present invention. FIG. 6A is a side perspective view of vascular port 44 with top screw cap 7 in place, and FIG. 6B is a cross sectional view of vascular port 44 without top screw cap 7. Referring to FIG. 6A, two sections of tubing 22 descend from the bottom of vascular port 44 which may connect vascular port 44 to the vascular system of a person in which vascular port 44 is implanted. As shown in FIG. 6B, a threaded region 23 near the top of vascular port 44 may enable top screw cap 7 to screw into place on vascular port 44. A set of cylindrical cavities 24 inside the walls of vascular port 44 may connect tubing 22 to tubing 12 to which a stent may be connected, as shown by way of example in FIGS. 3A and 3B. Threading 19 on the side of vascular port 44 that matches threading 16 of internal circular disk 10, may enable internal circular disk 10 to be threaded into place in vascular port 44.

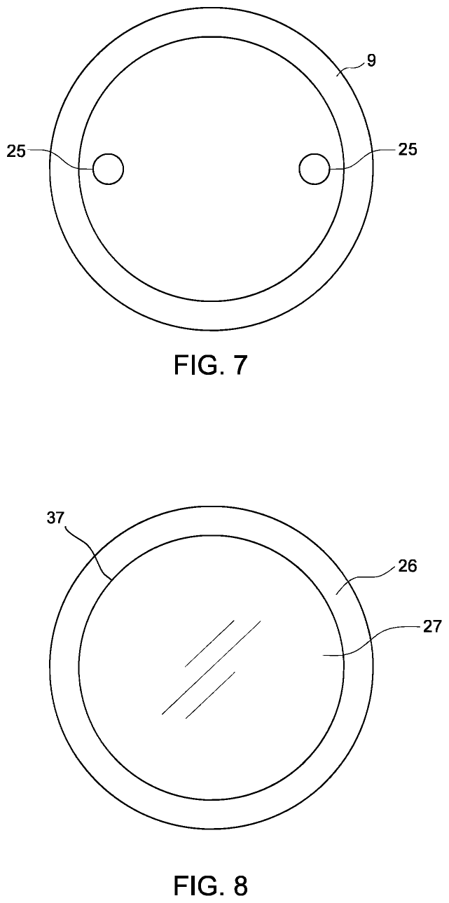

FIG. 7 is a bottom view of vascular port 44, in accordance with an embodiment of the present invention. In the present embodiment, vascular port 44 comprises two exit holes 25 in the bottom through which the lower region of tubing 22 exits.

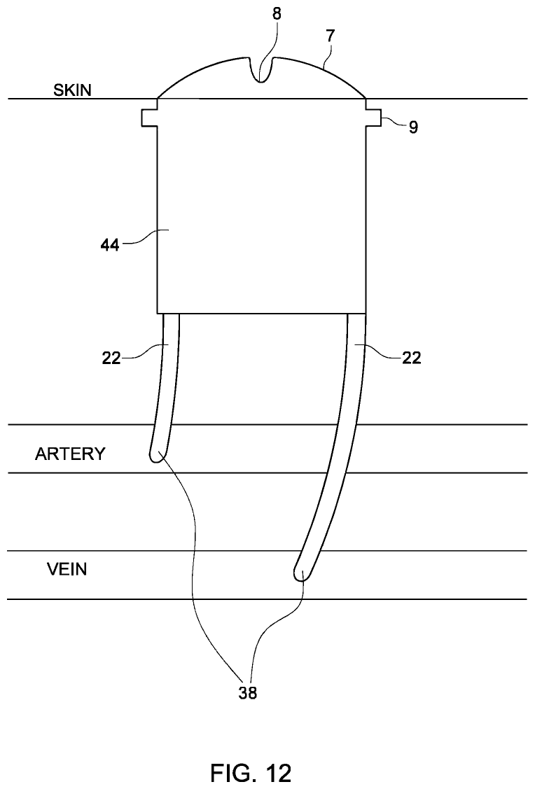

FIG. 8 is an interior bottom view of top screw cap 7, in accordance with an embodiment of the present invention. In the present embodiment, an overhang 26 may be disposed above a threaded extension 37 from the bottom of top screw cap 7. Threaded extension 37 may be screwed into threading 23 in the top of vascular port 44. When top screw cap 7 is screwed into place, overhang 26 may help create a tight closure between top screw cap 7 and vascular port 44. A washer or gasket may be used to enhance the seal between top screw cap 7 and vascular port 44, see FIG. 12 for example. A reflective surface 27 may be placed on the internal underside of top screw cap 7 for the purpose of reflecting light generated by an LED, see FIGS. 9A and 9B, onto the upper surface of the stent.

FIGS. 9A and 9B illustrate exemplary vascular port 44, in accordance with an embodiment of the present invention. FIG. 9A is a cross sectional view of vascular port 44 with interior components installed, and FIG. 9B is an exploded view of the components situated within the vascular port 44. In the present embodiment, a Light Emitting Diode (LED) 28 may be situated on top of circular depression 18 on internal circular disk 10. One wire 29 of LED 28 connects to a resistor 31 which in turn connects to a microcontroller 33. The other wire 30 of LED 28 connects directly to microcontroller 33. One of the voltage clamp wires 20 descending from voltage clamp 14a connects to a resistor 32 which in turn connects to microcontroller 33. The other voltage clamp wire 21 that is connected to voltage clamps 14b attaches to microcontroller 33 directly. A battery holder 34 connects to microcontroller 33 by two wires 35. Microcontroller 33 may be a small microcontroller such as, but not limited to, the commercially available “Beetle Board” which can be used to control the light, voltage, and magnetic impulses to the stent within the vascular port 44. Microcontroller 33 may also be programmed to allow the release of proteins/peptides into the bloodstream to be a timed release, which may help to ensure patient adherence to treatment regimens prescribed by physicians. Referring to FIG. 9B, a rubber washer 36 may be included that fits around the threading 37 on top screw cap 7 and rests against overhang 26 on top screw cap 7.

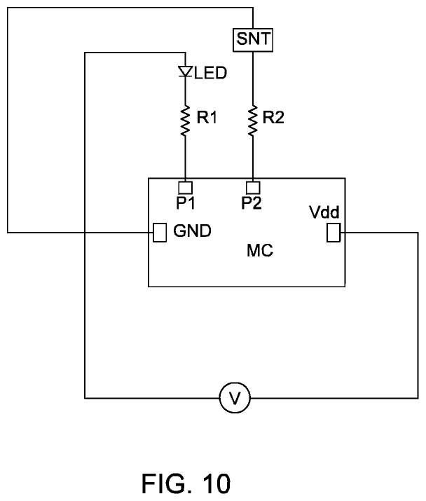

FIG. 10 is an electronic schematic for a microcontroller (MC) connected to a voltage/light operated version of an interchangeable stent for use in a vascular port, in accordance with an embodiment of the present invention. For example, this figure may represent the connection between microcontroller 33 and the stent shown by way of example in FIG. 9 in which the cells within the stent are activated by voltage or light. In the present embodiment, the microcontroller (MC) is connected to a battery (V) which attaches to the microcontroller (MC) at an IC power supply pin (Vdd) and a ground source (GND). A resistor 1 (R1) may connect from an Input/Output Source 1 (P1) to an LED and back to the ground source (GND). A resistor 2 (R2) may connect from an Input/Output Source 2 (P2) to the Tubular Metal Stent (SNT) and back to the ground (GND) source. Tubular Metal Stent (SNT) is made of metal in this embodiment to enable the voltage that may be stimulating the cells, for example endothelial cells, in Tubular Metal Stent (SNT) to be conducted through the body of Tubular Metal Stent (SNT).

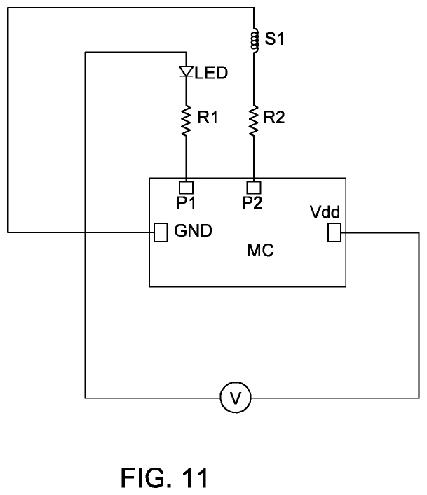

FIG. 11 is an electronic schematic for a microcontroller (MC) connected to a solenoid/light operated interchangeable stent for use in a vascular port, in accordance with an embodiment of the present invention. For example, this figure may represent the connection between microcontroller 33 and the stent shown by way of example in FIG. 9 in which the cells within the stent are activated by a solenoid or light. In this version, the body of the stent may be made of metal or plastic. In the present embodiment, the microcontroller (MC) is connected to a battery (V) which attaches to the microcontroller (MC) at an IC power supply pin (Vdd) and a ground source (GND). A resistor 1 (R1) may connect from an Input/Output Source 1 (P1) to an LED and back to the ground source (GND). A resistor 2 (R2) may connect from an Input/Output Source 2 (P2) to a solenoid (S1) and back to the ground (GND) source.

FIG. 12 is a perspective view of vascular port 44 implanted under skin and grafted to the vascular system, in accordance with an embodiment of the present invention. In the present embodiment, the lower regions 38 of tubing 22 that connect to the stent via cavities 24 in vascular port 44 and tubing 12 are grafted to an artery and a vein respectively. This creates an ateriovenous (AV) graft that connects to the stent through vascular port 44 to typically enable therapeutic proteins secreted by the monolayer of cells within the stent to enter into the bloodstream within the artery and the vein. It is contemplated that vascular port 44 would most likely be implanted near the clavicle with tubing 22 being inserted into the subclavian vein and and subclavian artery. However, vascular port 44 may be implanted in other areas of the body such as, but not limited to, the abdomen or peritoneum if needed or desired. In another embodiment, instead of an AV graft, the vascular port on both ends could connect to the same artery or on both ends to the same vein.

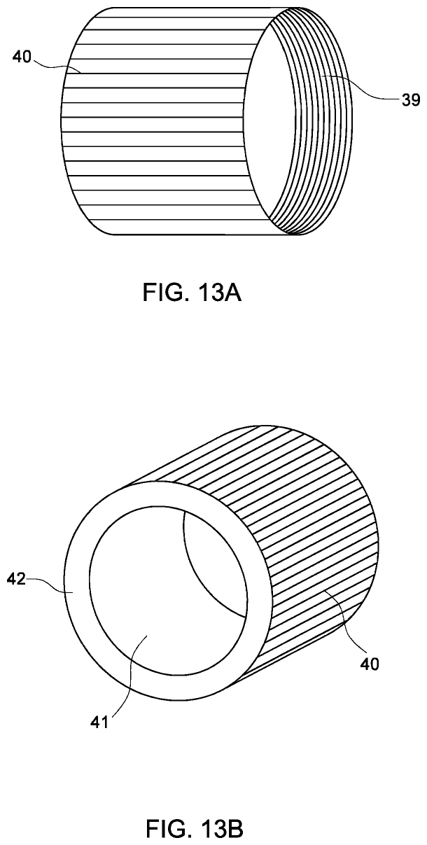

FIGS. 13A and 13B illustrate an exemplary screw connector 13, in accordance with an embodiment of the present invention. FIG. 13A is an exterior perspective right side view of screw connector 13, and FIG. 13B is an exterior perspective left side view of screw connector 13. Referring to FIG. 13A, in the present embodiment screw connector 13 may comprise threading 39 on its interior surface at one end, which may enable screw connector 13 to be threaded to threading on the body of a stent, see for example FIG. 1A and FIG. 1D. Referring to FIG. 13B, the opposite end of screw connector 13 may comprise a hole 41 surrounded by an outer lip 42. Hole 41 may enable tubing connected to the stent to pass through screw connector 13. A ribbed outside surface 40 composed of a set of evenly spaced ridges may be included to provide texture to aid in grip when tightening or loosening screw connector 13 from a stent.

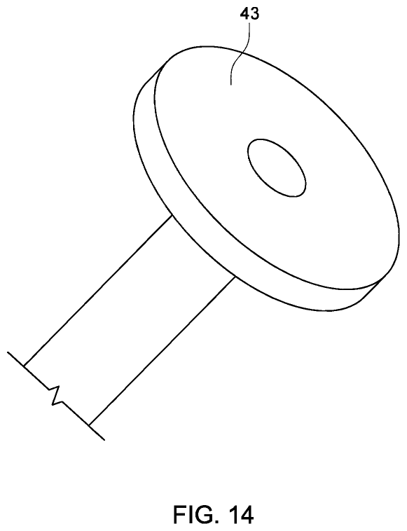

FIG. 14 is a perspective view of a terminal end 43 of the upper region of tubing 12, which may be connected to a stent, in accordance with an embodiment of the present invention. In the present embodiment, the terminal end of the upper region of tubing 12 contains a cylindrical outer edge 43.

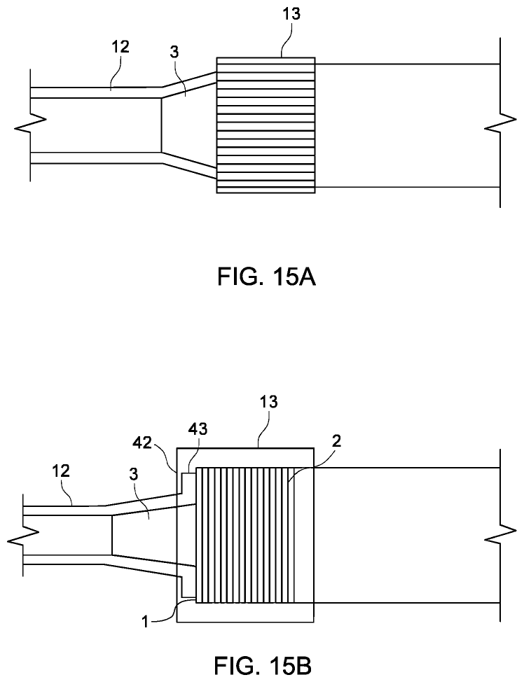

FIGS. 15A and 15B illustrate an exemplary connection between tubing and a tubular stent, in accordance with an embodiment of the present invention. FIG. 15A is a side view of the junction of the terminal end of tubing 12 to end nozzle 3 of main body 1 of the tubular stent, and FIG. 15B is a partially transparent side view of the same junction. Referring to FIG. 15A, screw connector 13 secures the terminal end of tubing 12 in place around end nozzle 3 of the stent. Referring to FIG. 15B in which screw connector 13 is shown as transparent to illustrate the junction within screw connector 13, cylindrical outer edge 43 of the upper region of tubing 12 fits into screw connector 13 between outer lip 42 of screw connector 13 and the end of main body 1 of the tubular stent. Tubing 12 may be made of a flexible material that may enable tubing 12 to stretch over end nozzle 3. Screw connector threads 39, shown by way of example in FIG. 13A, may screw into threads 2 on main body 1 of the stent. This type of junction typically provides a secure junction between the stent and tubing 12 in order to help prevent leakage

It is contemplated that various tubular stents according to embodiments of the present invention could be implanted without the use of a vascular port and left in the patient's body until the endothelial cells eventually die off. In another embodiment, a vascular port with an interchangeable stent system may be implemented without a microcontroller or any other electronics. This would allow the port to be smaller and more easily fit into the region around the clavicle. In yet another embodiment, the stent and the vascular port tubes may be capped with dialysis membranes. In this system, peptides could be secreted from the endothelial cells and pass through the dialysis membranes into the bloodstream without having blood actually pass through the stent at all. In yet another embodiment, the stent may be coated with a layer of chemical compound that can then diffuse over time into the bloodstream instead of a cell monolayer.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification. Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112(6) (post AIA 112(f)) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3rd parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing an implantable vascular port comprising an interchangeable stent-cartridge system according to the present invention will be apparent to those skilled in the art. Various aspects of the invention have been described above by way of illustration, and the specific embodiments disclosed are not intended to limit the invention to the particular forms disclosed. The particular implementation of the implantable vascular port comprising an interchangeable stent-cartridge system may vary depending upon the particular context or application. By way of example, and not limitation, the implantable vascular ports comprising interchangeable stent-cartridge systems described in the foregoing were principally directed to implementations involving therapeutic agents being released from cells within a stent; however, similar techniques may instead be applied to, systems releasing chemotherapy agents, other types of medications, radiation, or nutritional supplements which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre-AIA) or 35 USC 112(f) post-AIA. Otherwise, no limitations from the specification are to be read into any claims, unless those limitations are expressly included in the claims.

What is claimed is:

1. An interchangeable vascular stent cartridge, comprising:
an elongated hollow main body; and
said main body including threaded regions at distal ends of said main body, wherein said distal ends are capped with a nozzle at each terminal end of said main body.

2. The interchangeable vascular stent cartridge, as recited in claim 1, wherein said elongated hollow main body defines a cavity configured to receive a monolayer of genetically modified endothelial cells.

3. The interchangeable vascular stent cartridge, as recited in claim 2, wherein said monolayer of cells within said genetic mechanism is attached to an interior surface of said main body.

4. The interchangeable vascular stent cartridge, as recited in claim 3, wherein said interior surface is fenestrated forming a three-dimensional lattice structure.

5. The interchangeable vascular stent cartridge, as recited in claim 1, wherein an interior surface of said main body comprises a high-impact polystyrene.

6. The interchangeable vascular stent cartridge, as recited in claim 5, wherein said polystyrene surface is coated with a solution of fibronectin at a 1:100 dilution ration in phosphate buffered saline applied at a volume of 150 microliters per centimeter squared.

7. The interchangeable vascular stent cartridge, as recited in claim 1, further comprising a solenoid sheathing wrapping around an outer surface of said main body with a pair of cylindrical metal end pieces.

8. The interchangeable vascular stent cartridge, as recited in claim 2, wherein said monolayer of genetically modified endothelial cells is capable of responding to changes in wavelength and intensity of light.

9. The interchangeable vascular stent cartridge, as recited in claim 1, wherein said main body is constructed of a transparent material, whereby said transparent material allows light to influence the monolayer of genetically modified endothelial cells.

10. The interchangeable vascular stent cartridge, as recited in claim 1, wherein said main body is constructed of a conductive material, whereby said conductive material allows voltage to influence the monolayer of genetically modified endothelial cells.

11. The interchangeable vascular stent cartridge, as recited in claim 1, whereby said solenoid allows magnetic fluctuations to influence the monolayer of genetically modified endothelial cells.

12. The interchangeable vascular stent cartridge, as recited in claim 1, wherein said main body is constructed of a conductive transparent ceramic material, whereby said ceramic material allows light, voltage, and magnetism to influence the monolayer of genetically modified endothelial cells.

13. A vascular port system, comprising:
an interchangeable vascular stent cartridge;
a sealable vascular port configured to receive an interchangeable vascular stent cartridge;
a pair of voltage clamps for securing said interchangeable vascular stent cartridge to said vascular port;
said vascular port includes a screw cap to seal a top of said vascular port; and
said vascular port including a circular lip, wherein said circular lip holds the vascular port underneath a user's skin.

14. The vascular port system, as recited in claim 13, further comprising:
an internal divider in said sealable vascular port, whereby said divider is a circular disk with a threaded perimeter to engage with an internal threaded portion of said vascular port thereby separating said vascular port into an upper portion and a lower portion.

15. The vascular port system, as recited in claim 14, wherein said circular disk further includes surface depressions configured for grip to aid in the rotational securement of said circular disk.

16. The vascular port system, as recited in claim 14, wherein said pair voltage clamps are affixed to an upper surface of said circular disk, thereby securing said interchangeable vascular stent cartridge.

17. The vascular port system, as recited in claim 16, wherein said pair of voltage clamps connect to a pair of wires that descend to electronic components in a lower portion of said vascular port, wherein said electronic components include a microcontroller and battery pack.

18. The vascular port system, as recited in claim 14, wherein said circular disk includes a center hole, whereby a controllable light source controlled by a microcontroller extends through said hole.

19. The vascular port system, as recited in claim 13, further comprising a microcontroller disposed in a lower portion of said vascular port whereby said microcontroller initiates signals to at least one of the steps comprising toggling a light source, toggling a voltage through said voltage clamps, and toggling magnetic impulses though said solenoid.

20. The vascular port system, as recited in claim 19, wherein said microcontroller contains instructions to release proteins and peptides into a user's bloodstream on a time-release interval.

21. The vascular port system, as recited in claim 13, wherein said sealable vascular port includes a removable screw cap having a threaded lower portion for complemental engagement with a threaded portion on said vascular port and a washer or gasket to create a tight seal between said vascular port and an overhang on said screw cap.

22. The vascular port system, as recited in claim 21, wherein said screw cap is dome shaped with a groove in said screw cap for grip when fastening, and said screw cap includes a reflective lower surface to reflect light from a light source coupled to a microcontroller.

23. The vascular port system, as recited in claim 13, wherein said interchangeable vascular stent cartridge comprises:
an elongated hollow main body; and
said main body including threaded regions at distal ends of said main body, wherein said distal ends are capped with a nozzle at each terminal end of said main body.

24. The vascular port system, as recited in claim 23, further comprising:
a pair of flexible tubes connectable at one end to said nozzles of said interchangeable vascular stent cartridge;
a pair of threaded fasteners engaging with threads at said threaded regions of said main body of said interchangeable vascular stent cartridge, whereby said fasteners define a circumferential hollow tube with two open ends, internal threading, a ribbed outer surface, and an internal lip on a distal end of said hollow tube of said fastener to contain an outer edge of said flexible tube when said fastener threads on to said threading of a distal end of said interchangeable vascular stent cartridge thereby compressing the outer edge of said flexible tube;
wherein said threaded fasteners are screw connectors;
said pair of tubes comprise a flexible material to stretch over said nozzles and form a tight fit; and
said flexible tubes connecting at an opposing end to a pair of cavities in said vascular port, wherein a second pair of flexible tubes connect to an opposing end of said cavities and descend through holes in a bottom of said vascular port, whereby said second pair of tubes engage with at least one of an artery connection and a vein connection, a vein connection and a vein connection, and an artery connection and an artery connection.

25. The vascular port system, as recited in claim 24, wherein at least one tube in said pair of flexible tubes further includes a microfilter fluid bubble trap.

26. The vascular port system, as recited in claim 24, further comprising
a solenoid sheathing wrapping around an outer surface of said main body with a pair of cylindrical metal end pieces;
wherein said elongated hollow main body defines a cavity configured to receive a monolayer of cells with a genetic mechanism attached to an interior surface of said main body, in which said interior surface is fenestrated forming a three-dimensional lattice structure, and said interior surface comprises a high-impact polystyrene, wherein said polystyrene surface is coated with a solution of fibronectin at a 1:100 dilution ration in phosphate buffered saline applied at a volume of 150 microliters per centimeter squared;
wherein said monolayer of genetically modified endothelial cells is capable of responding to changes in wavelength and intensity of light; and
wherein said main body is constructed of at least one of: a transparent material, whereby said transparent material allows light to influence the monolayer of genetically modified endothelial cells, a conductive material, whereby said conductive material allows voltage to influence the monolayer of genetically modified endothelial cells, includes a solenoid to allow magnetic fluctuations to influence the monolayer of genetically modified endothelial cells, and a conductive transparent ceramic material, whereby said ceramic material allows light, voltage, and magnetism to influence the monolayer of genetically modified endothelial cells.

27. A method, comprising:
providing a vascular port system, comprising:
an interchangeable vascular stent cartridge having an elongated hollow main body, wherein said main body includes threaded regions at distal ends of said main body, and said distal ends are capped with a nozzle at each terminal end of said main body;
a sealable vascular port configured to receive said interchangeable vascular stent cartridge;
a pair of voltage clamps for securing said interchangeable vascular stent cartridge to said vascular port;
said vascular port includes a screw cap to seal a top of said vascular port;
said vascular port including a circular lip, wherein said circular lip holds the vascular port underneath a user's skin;
a pair of flexible tubes connectable at one end to said nozzles of said interchangeable vascular stent cartridge;
said flexible tubes connecting at an opposing end to a pair of cavities in said vascular port, wherein a second pair of flexible tubes connect to an opposing end of said cavities and descend through holes in a bottom of said vascular port, whereby said second pair of tubes engage with at least one of an artery connection and a vein connection, a vein connection and a vein connection, and an artery connection and an artery connection;
at least one of a light source, a voltage source, or a magnetic field source; and
implanting said vascular port system, wherein the flexible tubes and second pair of tubes of said vascular port system are configured to connect to at least a vein and at least an artery to provide for activating genes inside the monolayer of cells within the interchangeable vascular stent cartridge that in turn produce proteins that are secreted by the cells into the bloodstream.

28. The method as recited in claim 27, whereby once implanted, said vascular port system is configured to perform at least one of:
creating an electronically-controlled in vivo environment for genetically modified cells that can respond to changes in light by implementing a light source within said vascular port system that influences the generation of therapeutic proteins or peptides that can be secreted into the bloodstream, wherein said main body of said vascular stent cartridge is a transparent material;
creating an electronically-controlled in vivo environment for genetically modified cells that can respond to changes in electricity by implementing a pair of voltage clamps that secure said interchangeable vascular stent cartridge within said vascular port system that influences the generation of therapeutic proteins or peptides that can be secreted into the bloodstream, wherein said main body of said vascular stent cartridge is a conductive material; and
creating an electronically-controlled in vivo environment for genetically modified cells that can respond to changes in magnetic fields by implementing a solenoid to wrap around said interchangeable vascular stent cartridge within said vascular port system that influences the generation of therapeutic proteins or peptides that can be secreted into the bloodstream.

29. The method as recited in claim 27, whereby once implanted, said vascular port system is configured to execute a program on a microcontroller disposed in a lower portion of said vascular port system to allow the a timed release of proteins and peptides into the bloodstream by modulating the interval of pulses to a triggering mechanism, wherein said triggering mechanism is at least one of a light source, a voltage source, and a magnetic field, whereby once triggered, generated therapeutic proteins or peptides can be secreted into the bloodstream.

30. The method as recited in claim 27, further comprising:
configuring an interior surface of said interchangeable vascular stent cartridge as a high-impact polystyrene, wherein said polystyrene surface is coated with a solution of fibronectin at a 1:100 dilution ration in phosphate buffered saline applied at a volume of 150 microliters per centimeter squared; and
configuring an interior surface of said interchangeable vascular stent cartridge to be fenestrated to create a three dimensional lattice within the interchangeable vascular stent cartridge to increase the total surface area for cells to grow on thereby increasing the total amount of peptides secreted from the interchangeable vascular stent cartridge.
